(19) United States
(12) Patent Application Publication
(10) Pub. No.: US 2008/0049897 A1
(43) Pub. Date: Feb. 28, 2008

(54) System and Method for Temporally Precise Intensity Modulated Radiation Therapy (Imrt)

(76) Inventor: Janelle A. Molloy, Rochester, MN (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 11/597,073

(22) PCT Filed: May 24, 2005
(86) PCT No.: PCT/US05/18064

§ 371 Date: Nov. 20, 2006

Related U.S. Application Data
(60) Provisional application No. 60/573,895, filed on May 24, 2004.

(51) Int. Cl. A61N 5/10

(52) U.S. Cl. 378/65

(57) ABSTRACT

Radiation therapy or diagnostic that will ultimately be delivered in a manner that, in addition to being spatially precise, is capable of adapting instantaneously to changes in patient or subject anatomy. The related system and method can adapt to the time dependent geometry of internal patient anatomy and yield a temporally precise IMRT beam that is optimized for the instantaneous configuration of the internal target and avoidance structures.

### CROSS-REFERENCE TO RELATED APPLICATIONS

This application claims priority from U.S. Provisional Application No. 60/573,895, filed on May 24, 2004, entitled “System and Method for Temporally Precise Intensity Modulated Radiation Therapy (IMRT),” the disclosure of which is hereby incorporated by reference in its entirety.

### FIELD OF THE INVENTION

The present invention relates generally to a system and method for providing temporally precise intensity modulated radiation therapy (IMRT) or diagnostics, and more particularly to a system and method that can adapt to the time dependent geometry of subject's anatomy and yield a temporally precise IMRT beam that is optimized for the instantaneous configuration of the internal target and avoidance structures.

### BACKGROUND OF THE INVENTION

Intensity modulated radiation therapy (IMRT) has recently become feasible in many clinics. This technology produces highly conformal radiation patterns that allow physicians to treat tumorous tissues to high doses while sparing the surrounding healthy tissues. In conventional practice, this is implemented using linear accelerators equipped with mechanical “multi-leaf” collimators. This has been made feasible in part by the successful development of inverse treatment planning systems.

Conventional IMRT delivered via multileaf collimators requires 30-60 seconds to deliver per modulated field. As such, it is not capable of dynamically readjusting to changes in patient anatomy due to respiratory motion.

While the wide-spread acceptance of conventional intensity modulated radiation therapy (IMRT) has resulted in an increase in the spatial precision with which treatment can be delivered, respiratory-induced organ motion limits its effectiveness in many situations.

It should be appreciated that respiratory induced organ motion limits ones ability to apply conventional IMRT to lesions in the lung and abdomen. In conventional practice, compensation for this motion is achieved through the addition of large treatment margins or through the use of gating. Neither approach is ideal, as large treatment margins necessarily irradiate significant amounts of surrounding healthy tissues. While gating reduces these margins, it does not eliminate them and does so at the expense of increased treatment time.

### BRIEF SUMMARY OF INVENTION

Some embodiments of the present invention method and system alter the method by which the highly conformal radiation doses are delivered. By not using a moving, mechanical collimation system, some embodiments of the present invention will be able to, among other things, deliver a modulated radiation field that can track organ motion (due to, e.g., respiratory motion) in real time.

Some embodiments of the present invention method and system shall provide a small high energy photon beam that can be redirected precisely both in space and time, and can deliver a low dose, modulated radiation field in a time that is small compared to the respiratory cycle. By summing many such low dose fields, it will be able to compensate for changes in patient or subject anatomy, virtually instantaneously and can deliver a more highly conformal radiation dose, relative to the moving target and normal tissue structures. Some embodiments of the present invention method and system shall produce the scanning photon beam by magnetically redirecting a high energy electron beam in much the same way the electron beam is redirected in the cathode-ray tube in a television. This redirected electron beam will strike a target material that will produce a diffuse, but forward directed high energy photon beam. This beam will then be narrowed down to a “pencil beam” geometry via a unique double-focused collimation system.

An aspect of an embodiment of the present invention provides a system for irradiating a subject. The system comprising: a directed charged particle beam source for supplying charged particles; a scanning device for scanning the charged particles received from the source; a target, wherein the scanned charged particles impinges upon the target for supplying photons; and a collimator device that collimates the photons. The collimator device may be adapted to provide emerging radiation beams to the subject, wherein the emerging radiation beams are being sharply forward directed to the subject and with a small cross-section to form beamlets. The system may further include a control unit (or controller or processor) for generating signals for controlling the system or portions thereof.

An aspect of an embodiment of the present invention provides a system for irradiating a subject. The system comprising: a directed charged particle beam source for supplying charged particles; a scanning device for scanning the charged particles received from the source; and a collimator device that receives the charged particles received from the scanning device and collimates the charged particles as electrons. The collimator device may be adapted to provide emerging radiation beams to the subject, wherein the emerging radiation beams are being sharply forward directed to the subject and with a small cross-section to form beamlets.

An aspect of an embodiment of the present invention provides a method for irradiating a subject. The method comprising: supplying charged particle beams; scanning the charged particles received from the source; converting the scanned charged particles into photons; and collimating the photons to provide emerging radiation beams to the subject. The emerging radiation beams may be sharply forward directed to the subject and with a small cross-section to form beamlets.

An aspect of an embodiment of the present invention provides a method for irradiating a subject. The method comprising: supplying charged particle beams; scanning the charged particles received from the source; and collimating the charged particles as electrons to provide emerging radiation beams to the subject. The emerging radiation beams may be sharply forward directed to the subject and with a small cross-section to form beamlets.

These and other objects, along with advantages and features of the invention disclosed herein, will be made more apparent from the description, drawings and claims that follow.

### BRIEF SUMMARY OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 is a schematic illustration of an intensity map.

FIG. 2(A) schematically illustrates an application of an intensity map using DMLC in which target structure and critical structure volumes are stationary.

FIG. 2(B) is a simulation of respiratory induced motion in which the target structure moves to the left and the spinal cord or critical structure remains stationary.

FIG. 2(C) illustrates the application of temporally precise full intensity maps using the various embodiments of the present invention, wherein the scanning photon beam is in motion and target organ is stationary.

FIG. 3 schematically illustrates an embodiment of the present invention system that is capable of delivering temporally precise IMRT.

FIG. 4 provides a schematic drawing of the geometry associated with calculations of the required magnetic field strength.

FIG. 5 illustrates an exemplary embodiment of a first order collimator of the present invention, which illustrates the attenuation of two components of the incident photon beam as they pass through the collimator grid.

FIG. 6 illustrates an embodiment of the steering file components that drive of the electron beam of an embodiment of the present invention system.

### DETAILED DESCRIPTION OF THE INVENTION

It should appreciated that present invention radiation therapy will ultimately be delivered in a manner that, in addition to being spatially precise, is capable of adapting instantaneously to changes in patient or subject anatomy. The various embodiments of the present invention provide a system and method that can adapt to the time dependent geometry of internal patient anatomy and yield a temporally precise IMRT beam that is optimized for the instantaneous configuration of the internal target and avoidance structures.

Delivery of conventional IMRT is currently achieved via mechanical motion of multi-leaf collimators (MLCs). The modulation and delivery of each field requires 30-60 seconds and therefore is limited in its ability to respond to instantaneous changes in patient anatomy. However, regarding some embodiments of the present invention, there is provided an innovative treatment delivery system and related method in which modulation of the radiation beam is achieved electronically and is therefore capable of increasing the temporal precision by several orders of magnitude. Such embodiments may achieve this through the development of a unique scanning photon pencil beam and collimation system.

At least some embodiments of the present invention system and method provide the, ability to vary the entirety of the applied intensity map instantaneously, and can therefore deliver a modulated radiation field that is optimized for the specific target and structure geometry at any given moment in time. This will allow physicians to reduce treatment margins and pursue aggressive dose escalation. Further, the electronic steering mechanism will reduce maintenance requirements, as there will be no moving parts in some embodiments, yet in other embodiments there may be a combination of moving and non-moving parts. Accordingly, the present invention system may replace some or all standard mechanical leaf collimators MLCs, wedges and blocks used in conventional radiation therapy.

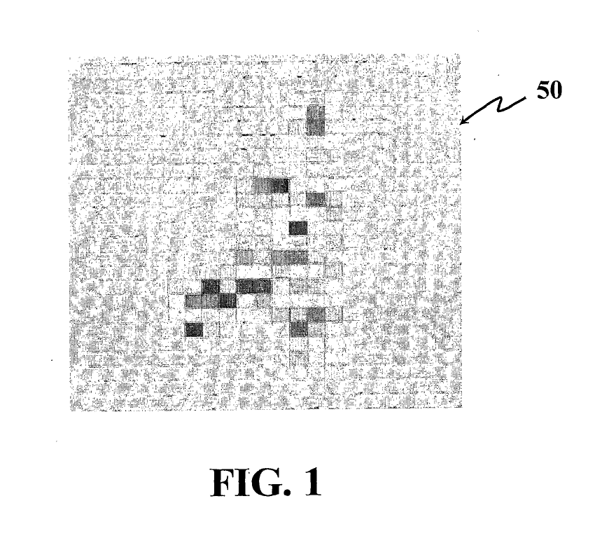

IMRT planning and delivery utilizes intensity maps 50, an example of which is illustrated in FIG. 1. These are produced by subdividing a radiation beam into small beamlets, typically about 5-10 mm in cross section. These intensity maps are optimized during the treatment planning process using three-dimensional anatomical information derived from CT or MR imaging. IMRT using dynamic MLC (DMLC) applies these intensity maps 50 by moving the mechanical leaves across the field, exposing small beamlets for different time intervals, depending on the intensity required at that point. The intensity map is delivered in a serial fashion as the leaves move from one end of the field to the other and requires 30-60 seconds. Using this conventional method, compensation for respiratory-induced motion is limited to the translation of the current beamlet in a single dimension.

The various methods and systems provided by the various embodiments of the present invention are inherently different. For example, some embodiments may deliver a given radiation field through various approaches. For instance, an aspect of an embodiment of present invention is to provide the superposition of many low dose fields. Each of these low dose fields will deliver the entirety of a fully modulated intensity map in less than about 500 ms for instance. It should be appreciated that the duration may vary as desired or required. For example, other exemplary durations may include, but not limited thereto, the following: less than 5,000 ms, less than 50 ms, less than, 5 ms, less than 100 minutes, less than 10 minutes, or less than 1 minute. The design of the instantaneously-applied low dose intensity map may be optimized for the patient geometry that exists at that point within the respiratory cycle or any other applicable cycle or predetermined period as desired or required. This is necessitated by the fact that target and normal structures do not all move as a rigid body during respiration or cardiac cycles.

Further, an aspect of an embodiment of present invention is to provide radiation treatment that will be delivered with the proposed invention system and method by serially applying and recording the beamlets in the appropriate intensity map. A feedback module, such as an external respiratory feedback (e.g., spirometry or fluoroscopy devices) will trigger switching to the succeeding intensity map. Full treatment will occur over multiple respiratory cycles, with the beamlet application for each intensity map resuming at the point from which it left off in the previous cycle. Because the beamlet switching is accomplished electromagnetically, the temporal precision of the present invention system is orders of magnitude greater than existing systems.

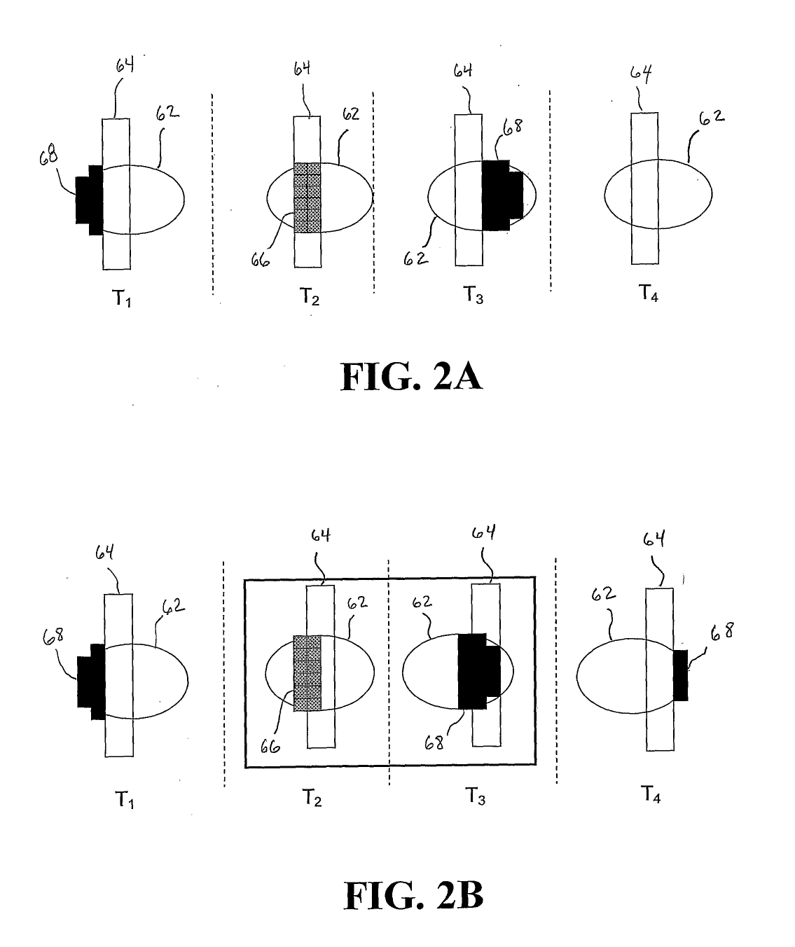

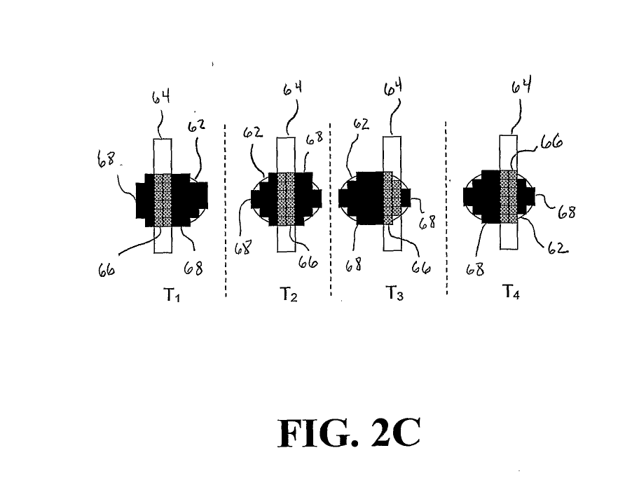

Referring to FIG. 2(A), FIG. 2(A) schematically illustrates an application of an intensity map using DMLC in which target structure 62 and critical structure 64 volumes are stationary. The irradiated beamlets move across the field and deliver a high dose, as illustrated by reference number 68 (e.g., black squares), to the target structure, as illustrated by reference number 62 (e.g., oval), and an optimized lower dose, as illustrated by reference number 66 (e.g., gray squares), to the region of the target structure 62 that overlaps the critical structure 64.

Next, referring to FIG. 2(B), FIG. 2(B) respiratory induced motion is simulated in which the target structure 62 moves to the left and the spinal cord or critical structure 64 remains stationary. By tracking the motion of target structure 62 and applying the beamlets relative to the moving target structure 62, one can appreciate that the resulting dose to the critical structure 64 exceeds that which was planned.

Next, referring to FIG. 2(C), FIG. 2(C) illustrates the application of temporally precise full intensity maps using the various embodiments of the present invention system and method. Here, the spinal cord or critical structure 64 may always receive a reduced dose, as illustrated by reference number 66 (e.g., gray squares), while the non-overlapping target receives full dose, as illustrated by reference number 68 (e.g., black squares). By optimizing and applying the entire dose over multiple respiratory cycles, the present invention system and method has the unique ability to take advantage of excursions that temporarily move the target structure 62 away from critical structures 64 and can even treat portions of the target structure 62 during periods in the respiratory cycle when they do not overlap critical structures 64. It should be appreciated that in addition to critical structures, other types of structures or regions may be avoided as desired or required. Some examples of critical structures may include, but not limited thereto, spinal chord, bowel, lung, heart, bronchi, esophagus and trachea.

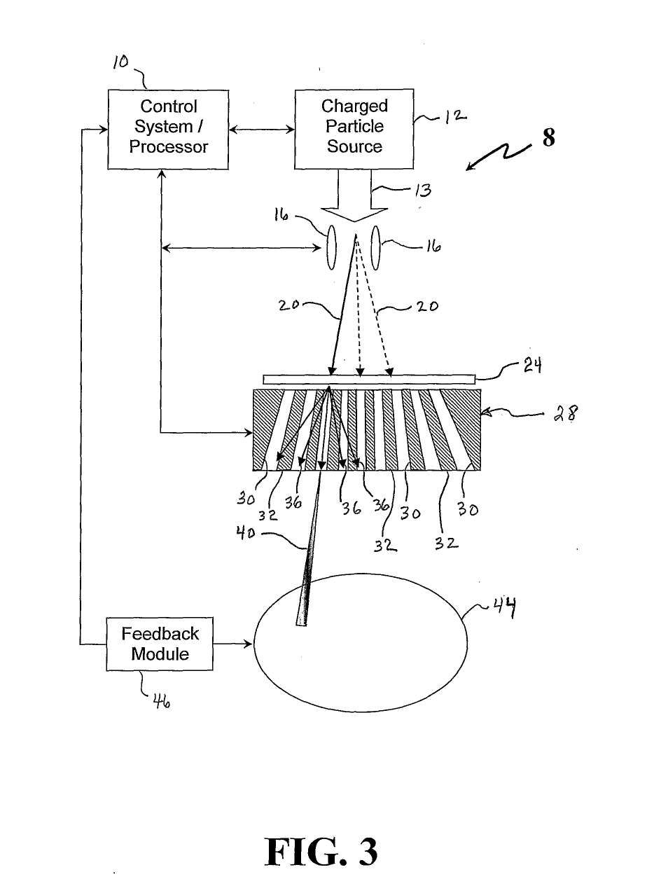

FIG. 3 schematically illustrates an embodiment of the present invention system that is capable of delivering temporally precise IMRT. The radiation system 8 for irradiating a subject 44 includes a directed charged particle beam source 12 for supplying charged particle beam(s) 13. Next, a scanning device 16 is provided for scanning the charged particles received from the source. In some embodiments a target 24 is provided, such as an extended target, wherein the scanned charged particles impinge upon the target 24, which in turn supplies photons 36. Further, a collimator device 28 collimates the photons, whereby the collimator device is adapted to provide emerging radiation beams 40 to the subject 44 of interest. The emerging radiation beams 40 may be sharply forward directed to the subject and with a small cross-section to form beamlets. The radiation system 8 further comprises a control unit or computer processor 10 for generating signals for controlling said radiation system 8 or components thereof. It should be appreciated that the subject 44 may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have radiation therapy or diagnostics similar to a human (e.g., rat, dog). It should be appreciated that the subject may be, for example, any applicable patient.

In an embodiment of the present invention, a high energy electron beam(s) 13 (e.g., about 12 MeV, other ranges are available as desired or required) may be produced by a standard linear accelerator wave guide and accelerator tube 12. This thin beam(s) 13 may then be magnetically redirected via a two-dimensional electromagnetic coil system 16 in much the same way that the cathode ray is redirected in a television. The successful scanning of such a high-energy electron beam was proven feasible by the scanning electron beam accelerators that were commercially available in the 1980's. Although they were technically feasible, their production ceased, as they did not offer a compelling advantage over electron beams that incorporated a scattering foil. The scanned electron beam(s) 20 will then impinge upon a target 24 that is optimized in terms of geometry and material to yield a highly forward peaked x-ray beam. This target 24 may be referred to as the “extended” target. The bremstrahlung interaction that occurs in the target 24 will then yield a diffuse, but generally forward directed high-energy x-ray beam 36. (Note that in this context, “forward directed” refers to the instantaneous direction of the scanned electron beam.) The absence of a flattening filter will render the emerging x-ray beam more forward directed than the diffuse and uniform x-ray beams that typify commercial linear accelerators.

The diffuse, forward directed photon beam 36 will then be collimated by a unique, double focused collimation grid 28. By fabricating a collimation grid 28 with its divergence matched to the divergence of the scanned electron beam in both directions, the emerging high-energy photon beam 36 will be sharply forward directed and small in cross section, i.e. about 1 cm or less, of the subject, patient or animal.

In an embodiment, the present invention electron scanning strategy also comprises a highly novel component of this proposal. Instead of scanning the electron beam uniformly in order to produce a dosimetrically flat radiation field, the present invention scanning system and method may include repeated repositioning and dwell cycles in order to produce an intensity modulated radiation field. The present invention provides for required designing and calibrating of the control circuitry for the electromagnets directing the field. It should be noted that the present invention system may be implemented without any mechanical moving components. As such, the electronic control and application of the individual radiation pencil beams can be achieved in a time period that is instantaneous compared to respiratory-induced organ motion.

Although a full therapeutic dose will still require 30-60 seconds to deliver, in one embodiment of the present invention system and method, this will be accomplished via the application of many low-dose, intensity modulated fields. In this unique strategy, each low-dose field may be optimized for the instantaneous anatomical geometry that exists at the appropriate point in the respiratory cycle. In this manner, the applied modulated field will track the patient motion in real time and deliver an optimized radiation pattern, taking into account the full relative motion of target and normal structures.

Various embodiments of the present invention system will require real time feedback so that the steering system knows at what point in the respiratory cycle the patient is in. Several options exist, including, but not limited thereto spirometry, optical tracking of infrared emitters, fluoroscopic tracking of implanted markers, or direct tracking of organ motion via real time imaging. It should be appreciated that the while the feedback module 46 may be utilized for tracking respiratory characteristics, it may be utilized for tracking or monitoring any desired, required or inherent characteristic or feature of a subject, patient or animal, such as cardiac characteristics.

It should be appreciated that various embodiments of the present invention system and method include considerations on electronic timing relative to the temporal characteristics of the respiratory or cardiac cycle, required magnetic field strength, electromagnet design, basic power requirements and preliminary collimator design.

An aspect of the various embodiments of the present invention provides a unique treatment planning strategy capable of taking advantage of the unique treatment delivery capabilities of the present invention systems/methods. Specifically, because the present invention system/method can “instantaneously” switch from one intensity map to another, application of different intensity maps optimized for the relative, changing and deforming geometry of the anatomical structures is uniquely provided for the present invention system/method. As such, the present invention treatment planning system (TPS) or method yields a series of optimized intensity maps corresponding to the relative anatomical geometry that exists at a given point in the respiratory or cardiac cycle. As such, the output of the TPS will yield a series of intensity maps for each linear accelerator gantry angle.

For example, but not limited thereto, an embodiment of the present invention TPS will use deformable anatomical models and a four dimensional imaging modality (for example, 4D CT scanning). The output from the TPS may include a control file which is readable by the scanning photon pencil beam delivery device. This file will drive the electromagnetic steering system. One embodiment of this control file may include a series of electromagnet current values and associated dwell times.

Next, it should be appreciated that the radiation system may be in hard-wire or wireless communication with a computer processor(s)/controller(s) or user(s). Moreover, any one or all of the modules of the radiation system may be in communication with (hard wire or wireless) a remote processor/controller, communication device, and/or remote device via communication path or channel. Any part of the communications paths/channels (i.e., communication among the various modules illustrated in FIG. 3 as well as remote modules not specifically illustrated) and related may be implemented using wire or cable, fiber optics, a phone line, a cellular phone link, an RF link, an infrared link, and other physical or wireless communications channels, for example. Additionally, it is feasible that the various modules may be portable or in communication with a portable device such as a PDA, hand held device or lap top or embodied at a work station such at a personal computer or the like. It should be appreciated that any readings/data received or provided the radiation system may be uploaded via the internet for processing or as desired and sent back to the radiation system or any other location (local or remote) for use, processing or data storage for example.

An example of a remote device may be, for example, a display interface that forwards and provides graphics, text, and other data. Another example of a remote device may be memory, storage drive, or storage medium, such as a floppy disk drive, a magnetic tape drive, an optical disk drive, a flash memory, etc. Examples of a communications interface may include a modem, a network interface (such as an Ethernet card), a communications port (e.g., serial or parallel, etc.), a PCMCIA slot and card, a modem, etc. It should be appreciated that software and data transferred via communications interface or any portion of communication path or channel or the like may be in the form of signals which may be electronic, electromagnetic, optical or other signals capable of being received by communications interface as well as the radiation system, processor/controller, remote processor/controller, communication device, and/or remote device/system. Other examples of a remote device/system may include at least one of the following: personal computer, processor, keyboard, input device, mouse device, PDA, hand-held device, monitor, printer, work station, remote laboratory, remote medical facility, inpatient facility or system, outpatient facility or system, remote clinic, remote subject or patient site, internet system and intranet system.

It should be appreciated that any or all of the radiations system modules, processor, remote processor, communication device, remote device/system, and communication path or channel may be separately or integrally formed with one another. Moreover, any of these modules/components may be detachable, replaceable, stationary and portable.

Electronic Timing:

In an exemplary embodiment, the response of the system may be such that the time required delivering a full-field, modulated dose increment is small compared to the duration of a respiratory cycle. Assuming that the respiratory cycle duration is on the order of about 5 seconds, then the modulated dose increment must be delivered in less than about 0.5 seconds. Typical linear accelerators do not produce radiation continuously, but rather in pulses. The duty cycle of a typical linear accelerator is approximately 0.001. Specifically, the VARIAN 2300 CD has a pulse length of 7 μs and a distance between pulses of 2.4 ms, yielding a duty cycle of 0.003 at a dose rate of 400 monitor units (mu) per minute using a 6 MV photon beam. This may be measured using an oscilloscope connected to the reflected power terminal on the linear accelerator's control panel. One may then consider that the pulse rate is 1/(2.4 ms) or 416 Hz. It may be required that the transition time to steer the beam to the next beamlet is small compared to the pulse rate, or <2.4 ms. Thus, it may be required that steering between beamlets be accomplished in <0.2 ms or approximately 200 μs.

In an example, it may be set forth that the linear accelerator can deliver 416 pulses per second. The desired dose increment delivery time is 0.5 s as described above. Therefore, the linear accelerator can deliver 208 pulses per dose increment. Also, a beamlet intensity gradation of at least five may be specified, that is, the intensity of each beamlet ranges from 0 to 100% intensity in 20% increments. Since only a subset of the potential beamlets in any given field would require full intensity, and some would require zero intensity, we can approximate that, on average, each beamlet will require 50% intensity. It should be appreciated that the pulsed nature of the radiation requires that the minimum beamlet dose will consist of one pulse. Under these constraints, this approach is able to deliver a temporally precise, fully modulated field that is 9 cm×9 cm in dimension. That is, a 9×9 cm field possesses 81 potential beamlets. The number of pulses per dose increment will be equal to the number of potential beamlets (81) multiplied by the number of gradations (5) and the average intensity (50% or 0.5), or 81×5×0.5=202 pulses.

It should be appreciated that for certain applications of practicing the present invention method and system, the field size should be sufficient for many lung nodules and other lesions. If a larger region requires treatment, it is possible to simply divide the total irradiated area into segments that are 9×9 cm² or less and treat them sequentially. This approach is similar to the use of carriage shifts that the VARIAN MLC uses to treat IMRT fields that are larger than 14.5 cm in width. Ultimately this limitation may be circumvented in several ways. As linear accelerator manufacturers adopt the technology, it may be feasible to increase the pulse frequency. Accordingly, this will increase the field size of the dose increment that can be delivered in 500 ms.

An alternative delivery strategy of an embodiment includes serially applying and recording the beamlets in the appropriate intensity map. External respiratory feedback (e.g., spirometry or fluoroscopy) will trigger switching to the succeeding intensity map at an appropriate time. Full treatment will occur over multiple respiratory cycles, with the beamlet application for each intensity map resuming at the point from which it left off in the previous cycle. Because the beamlet switching is accomplished electromagnetically, the temporal precision of the system is orders of magnitude greater than existing systems. This strategy reduces the temporal constraints described above to simply being able to switch from one beamlet to another in a time period that is small compared to the respiratory cycle, i.e., 500 ms or applicable duration.

Power Requirements

Many of the various strategies of some of the embodiments of the present invention shall require power output from the linac (linear accelerator) that exceeds that which is typically available. However, benchmark considerations demonstrate that the power requirements for some embodiments of the present invention system can be met with minimal modifications to existing accelerator design. The required power can be calculated as follows.

Total number of beamlets per treatment=4200 A survey of a head and neck IMRT program indicated that a typical large neck plus anterior yoke field is delivered with approximately 150, 1 cm² beamlets. A 5 mm beamlet resolution would require 600 beamlets. These IMRT treatments are delivered using 7 separate gantry angles. Thus, the total number of beamlets needed to treat these large head and neck fields is 4200.

Output required per beamlet=155 cGy/s It is clinically reasonable to deliver an IMRT treatment in 15 minutes or less. Therefore the system must be able to deliver 4200 beamlets in 900 s, or 4.7 beamlets/s. A typical radiation dose is 200 cGy. When applied over 7 separate fields, each field must deliver 29 cGy, and since the beamlets in each field are delivered serially, they each must deliver 29 cGy to isocenter. The percent depth dose for a 5×5 cm, 15 MV field is approximately 0.859 at 10 cm depth. Therefore each beamlet must produce 33 cGy at dmax. The total required output is 33 cGy/beamlet×4.7 beamlets/s, or 155 cGy/s.

Available output without significant modification=100 cGy/s Existing linear accelerators (linacs) can produce a radiation output of 600 cGy per minute (10 cGy/s) at the depth of maximum dose for a 6 MV beam. An increase in energy by a factor of two will increase the bremsstrahlung efficiency by a factor of approximately four. Thus, a dose rate of 40 cGy/s is feasible for a 15 MV beam. Removal of the flattening filter will eliminate the absorption of approximately 60% of the beam, thereby increasing the available output to 100 cGy/s. Thus, increases in the power requirements over existing linacs will be moderate.

In addition, judicious restructuring of the intensity maps may provide significant additional precision in the following manner. Consider that there will be approximately 10 separate intensity maps to be delivered at various points in the respiratory cycle. Although these maps will differ from one another, they will possess many beamlets in common. That is, a beamlet that is being delivered in one intensity map is likely to be delivered the other intensity maps, even if not with exactly the same intensity. As such, the treatment can be divided into a static component and a dynamic component. The static component is composed of all beamlets that are irradiated in all intensity maps. The intensity of each of these static beamlets corresponds to the intensity map that requires the least intensity for that beamlet. Thus, this static component represents the part of the intensity map (both in geometry and dose) that is delivered during all points in the respiratory cycle. The dynamic component is then simply the difference between a specific intensity map and the static component. With the treatment plan subdivided in this manner, the static component could be delivered first without temporal precision. The dynamic component would then contain significantly fewer beamlets and intensity gradations, and could be delivered to a larger region with good temporal precision.

Magnetic Field Strength:

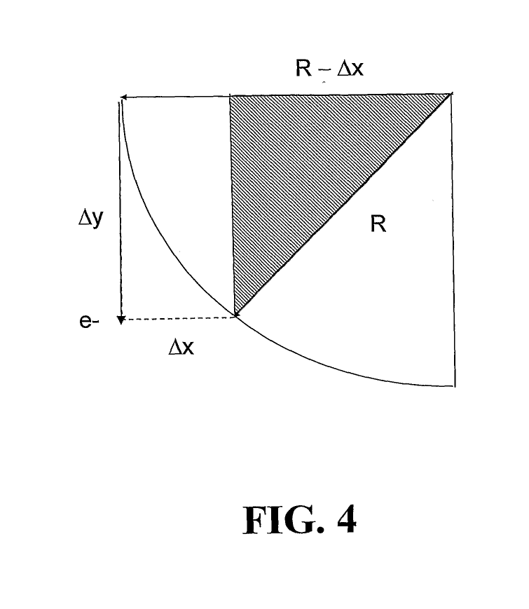

By equating the centripetal force (F_{c}) on a mass m, traveling at velocity v, in a circular path of radius R (F_{c}=m v²/R) to the force (F_{B}) acting on an electron of charge q in a magnetic field B (F_{B}=q v×B), the magnetic field strength required to deflect an electron can be written simply as B=mv/qR. A schematic diagram of the deflection geometry is shown in FIG. 4. Δy is the vertical travel path from the accelerator window to the extended target and Δx is the desired deflection. The path radius R can be solved in terms of Δy and Δx by applying the Pythagorean theorem to the right triangle indicated in the diagram. That is

$$R = \frac{Δ {y}^{2} + Δ {x}^{2}}{2 Δ x} .$$

For a vertical travel path Δy=10 cm and a deflection Δx=1 cm, and applying the values m=1.8×10⁻²⁹ kg (for a 10 MeV electron), v≈3×10⁸ m/s, and q=1.6×10⁻¹⁹ C, we find that the required magnetic field strength B, is 675 gauss (G) per 1 cm of deflection. Because of the magnification of the field size at the nominal source-to-axis distance of 100 cm, a 1 cm deflection at the target corresponds to approximately 10 cm at the patient and due to symmetry, a 20 cm wide field.
Electromagnet Design:

The magnetic field at a point z, along the axis of a loop of radius ρ, carrying current I is given by

$$B = \frac{{μ}_{o} I {ρ}^{2}}{{2 [ {ρ}^{2} + {z}^{2} ]}^{3 / 2}} ,$$

where μₒ=1.26×10⁻⁶ H/m is the magnetic permeability of free space. Solving for I and assuming that ρ=z=3 cm, we determine that I=9×10³ A. This value can be effectively achieved by using multiple turns in the coil and inserting a “mu metal” into the core of the electromagnet. The magnetic field scales linearly with the number of turns in the coil and the relative permeability μᵣ of its core. Thus, a 100 turn electromagnet with a core possessing a μᵣ=100 could deliver the required magnetic field with a current of 0.9 A. All of these values are completely feasible, as mu metals possessing permeabilities of 10,000 or more are readily available.

Electromagnets possess a property referred to as inductance (L), that produces a time delay between the application of a voltage and the current running through the coil. This delay is described by a time constant τ_{L}=L/R, where R is the series resistance of the electromagnetic circuit. The time constant for the electromagnets in this system must be fast compared to the beamlet delivery time. Thus, τ_{L} must be on the order of 200 μs. The inductance of a coil is given by

$$L = \frac{N A B}{I} ,$$

where A is the cross sectional area of the coil and N is the number of turns. For the 100 turn coil described above, A=πρ²=(3.14) (0.03m)²=0.003 m². If B=675 gauss (=0.0675 T), and I=0.9 A, then L=0.02 H. Thus the time constant of 200 μs can be achieved if R=100 Ω. The power dissipation in such a coil would be equal to I²R or about 81 W. This is equivalent to the heat generated by 1 standard household light bulb. Thus, minimal cooling may or may not be necessary, but is clearly feasible in either case.
Dual Focused Collimator Design:

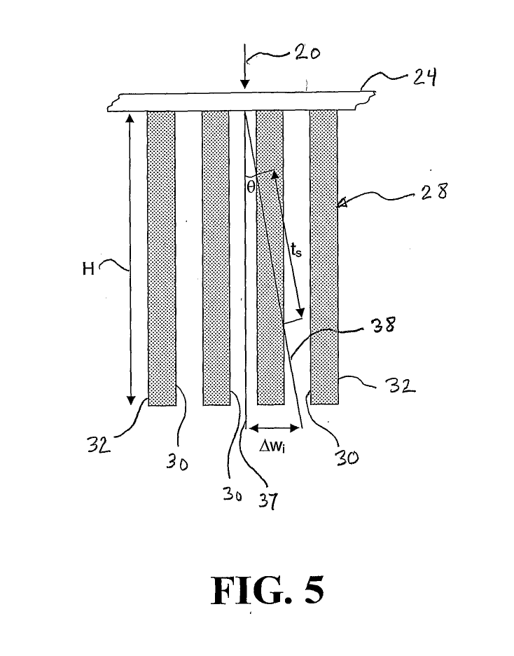

FIG. 5 illustrates an exemplary embodiment of a first order collimator of the present invention, which illustrates the attenuation of two components of the incident photon beam 36 as they pass through the collimator grid 28. It should be noted that the drawing is not drawn to scale and only illustrates one out of the two dimensions that it possesses. Because an attenuation of 5 half-value-thicknesses (HVT) decreases a radiation beam by 97%, and the effective “filling factor” of the collimator is ½, the total height, H, of the collimator may be chosen to be ten HVTs. For a 6 MV photon beam this is equal to 10×0.67″ of lead, or 17 cm. In this exemplary design the extended target is placed 10 cm from the incident electron beam source (e⁻ source) and the e⁻ source-to-treatment distance is 100 cm. The width of an exposed collimator “tunnel” 30 is therefore 1 mm in order to yield a 1 cm pencil beam at the treatment distance. Assuming the attenuator width to be equal to the tunnel width, the tunnel-to-tunnel spacing is then Δwᵢ=2 mm. It should be appreciated that other dimensions and sizes may be implemented as required or desired for given applications.

Still referring to FIG. 5, the central vertical line represents the forward-directed component 37 of the photon beam 36 that passes unattenuated through the collimator 28 and forms a 1 cm pencil beam at a distance of 100 cm. The angled line represents a divergent component 38 of the beam that is affected by one attenuator and exits the collimator 28 through the tunnel 30 adjacent to the forward-directed component 37. Trigonometric considerations imply that, tan θ=Δwᵢ/H, and that the attenuation thickness t_{S} is given by t_{S} sin θ=Δwᵢ/2. Given that Δwᵢ=2 mm and H=170 mm, θ is found to be 0.012 radians, and t_{S}=85 mm, or 5 HVTs. Thus, the photon component (reference 40 as shown in FIG. 3) exiting the collimator 28 at the adjacent tunnel 30 is attenuated by 97%. The same calculation can be extended to a photon component exiting the collimator at a point that is horizontally distant from the tunnel that defines the forward-directed photon beam. For example, at a distance of ten tunnel-attenuator widths, or 10 Δwᵢ, tan θ=10*Δwᵢ/H=(10*2 mm)/170 mm, and t_{S}=10*(Δwᵢ/2)/sin θ or t_{S}=85 mm. Again, this is equal to 5 HVT and will attenuate the beam by 97%. A further reduction in the dose at points that are not forward-directed can be expected based on the inherently forward-directed nature of the photon beam and the lack of a flattening filter.

Thus, the dual-focused collimator will effectively attenuate all components of a photon beam that are not forward-directed. Note again, that the position and direction of the incident electron beam 20 will be varied electro-magnetically over the range of the extended target 24, and that, in this context, the term “forward-directed” is defined by the instantaneous electron beam and its associated tunnel. It should be appreciated that the collimator design as described will yield a radiation field that possesses “dead spots” in the direct path of the attenuators in a pattern similar to a checkerboard. The various systems and methods of the present invention may, for example, address the issue of these dead spots through penumbra matching or by a single translation of the collimator mid-way through the treatment.

In this context, the term “penumbra matching” implies that the value of the dose from a single pencil beam will range from about 100% to 50% at the center of the irradiated tunnel and adjacent attenuator, respectively. There are several factors that will contribute to the smearing of a given beamlet and the resulting dose in the patient. These include scatter within the patient, scatter from the collimator, incomplete attenuation in the corners of each attenuator, superposition of irradiated fields from other gantry angles and the finite size of the photon focal spot. Variables over which we may be controlled in the collimator design include the “filling factor”, (i.e., the ratio of the width of the tunnel to the width of an adjacent attenuator) and the attenuator geometry. For example, it may be advantageous to taper the distal ends of the attenuators in order to increase the penumbra.

With regards to an exemplary embodiment for the fabrication of the present invention complex dual-focused collimator, the collimator may comprise an attenuator and tunnel configuration by stacking a series of flat pieces that possess precisely drilled holes. By varying the width and spacing of the holes in each consecutive piece, a full collimator can be fabricated with any desired divergence, filling factors, or attenuator tapering that is necessary. For example, three flat pieces that have had holes drilled can be provided to create a divergent tunnel pattern. In other embodiments, for example, it shall be possible to stack 10 to 200 such pieces or other levels of stacking as required or desired. Further, they may be fabricated from either lead or tungsten (or other suitable material), and the holes may be simple round drilled holes or they may be punched to form square cross sections. Other types of apertures, grooves, tunnels, passage ways, via, or conduits may be utilized as required or desired according to the given application. Each flat piece may be anchored precisely via four guide holes that will be drilled in each corner and through which four guide posts will protrude. Any suitable attachment or securing mechanism or material may be utilized. The step-wise nature of the cross section of each tunnel may at first appear undesirable. However, in some embodiments this shall prove to be advantageous in that it will smear the edge of the pencil beam and aid the penumbra matching constraint.

In an embodiment, if the penumbra matching is provided, then tunnel width may be about 0.5 mm. This will yield a tunnel-attenuator center-to-center distance of 1 mm at the collimator and 1 cm at the treatment distance. Further, if penumbra matching is not provided then other alternative strategies may be implemented. For example, one strategy utilizes the resulting radiation pattern's checkerboard appearance. By translating the collimator assembly by one pencil beam width midway through the treatment, a compensating intensity pattern will be created. This will require the design and fabrication of a motorized or pneumatic device, and while this may not be optimal in some situations, it would resolve the problem of dead spots.

A second alternative, for example, would be to compensate for the dead spots during treatment planning. Instead of optimizing each intensity modulated field assuming that all beamlets in a given field are available, one may simply exclude beamlets that correspond to the dead spots. While this may not be an optimal solution in some situations, scatter from adjacent exposed pencil beams and the superposition of modulated fields from additional gantry angles have the potential to compensate for dead spots. Consider too that ultimately, a full clinical implementation of the system may combine good, but incomplete, penumbra matching with compensation during treatment/planning much in the same way that current inverse treatment planning systems account for interleaf leakage and tongue and groove effects.

Electronic Input

The various embodiments of the present invention shall convert the intensity maps into input for the steering system. Such resulting input shall be referred to as the “steering file”. First, the method may include may include calibrating the conversion between pencil beam position and coil current. This conversion will be applied to the spatial positions of the beamlets in each of the intensity maps. The fluence in each beamlet is given by the dwell time of the deflected electron beam at that position. The calibration of dose per unit dwell time in each pencil beam will be calibrated using standard radiation therapy dosimetry techniques.

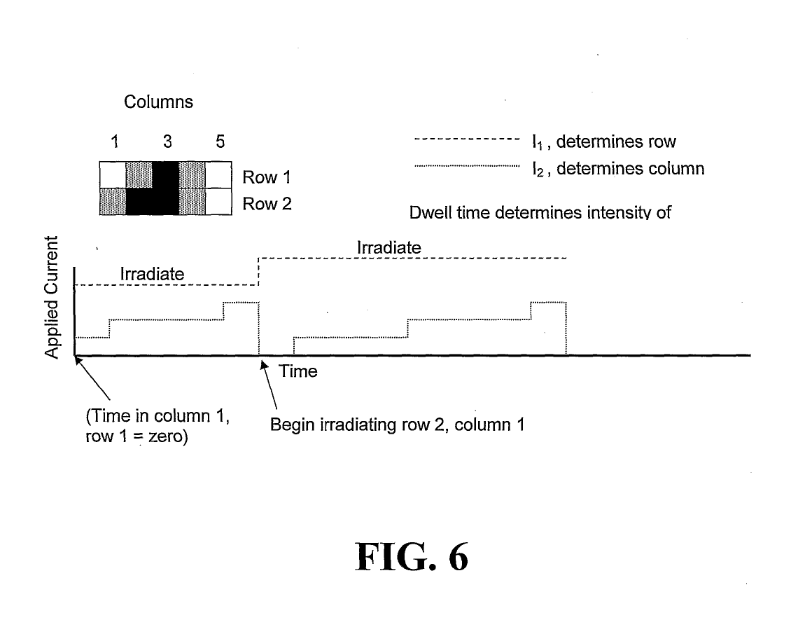

The steering file may consist of a series of coil currents and associated dwell times. FIG. 6 illustrates an embodiment of the steering file components that drive of the electron beam of an embodiment of the present invention system. The beam will be scanned along one axis, dwelling as necessary at each beamlet in a given row, and then advanced to the next row. This will be repeated until all of the rows have been irradiated. As described previously, the entire treatment will consist of the superposition of many “dose increments”. The completion of the scanning of all rows and columns corresponding to one intensity map will comprise one dose increment and will be completed in less than about 0.5 seconds, or as desired or required. Upon completion of one dose increment, the system will commence irradiation of the dose increment and associated intensity map that corresponds to the next appropriate point in the respiratory cycle. This cycle will be repeated until the sum of the dose increments that have been irradiated for each intensity map is equal to that planned.

In summary, some embodiments of the present invention method and system may be utilized for, but not limited thereto, radiotherapy linear accelerators and small animal experimental radiation treatment devices. Some embodiments of the present invention method and system may provide, but not limited thereto, the following advantages: temporally precise IMRT that can track organ motion, including respiratory and cardiac motion; replacement of mechanical multileaf collimators; provide IMRT treatments without any moving parts or less moving parts; and increase spatial precision of IMRT delivery.

The various embodiments of the present invention system and method may be implemented with the following exemplary radiation systems/methods, subsystems/sub-methods, external systems/methods, or external subsystems/sub-methods as disclosed in the following U.S. patents and of which are hereby incorporated by reference herein in their entirety:

U.S. Pat. No. 6,879,715 B2, to Edic, et al., entitled “Iterative X-ray Scatter Correction Method and Apparatus;”

U.S. Pat. No. 6,785,360 B1, to Annis, entitled “Personnel Inspection System with X-ray Line Source;”

U.S. Pat. No. 6,778,636 B1, to Andrews, entitled “Adjustable X-ray Beam Collimator for an X-ray Tube;”

U.S. Pat. No. 6,662,036 B2, to Cosman, entitled “Surgical Positioning System;”

U.S. Pat. No. 6,528,797 B1, to Benke et al., entitled “Method and System for Determining Depth Distribution of Radiation-Emitting Material Located in a Source Medium and Radiation Detector System for use Therein;”

U.S. Pat. No. 6,405,072 B1, to Cosman, entitled “Apparatus and Method for Determining a Location of an Anatomical Target with Reference to a Medical Apparatus;”

U.S. Pat. No. 6,402,373 B1, to Polkus et al., entitled “Method and System for Determining a Source-to-Image Distance in a Digital Imaging System;”

U.S. Pat. No. 6,396,902 B2, to Tybinkowski et al., entitled “X-ray Collimator;”

U.S. Pat. No. 6,393,100 B1, to Leeds et al., entitled “Asymmetric Collimator for Chest Optimized Imaging;”

U.S. Pat. No. 6,356,620 B1 to Rothschild et al., entitled “Method for Raster Scanning an X-ray Tube Focal Spot;”

U.S. Pat. No. 6,009,146, to Adler et al., entitled “MeVScan Transmission X-ray and X-ray System Utilizing a Stationary Collimator Method and Apparatus;”

U.S. Pat. No. 5,859,893 to Moorman et al., entitled “X-ray Collimation Assembly;

U.S. Pat. No. 4,726,046 to Nunan, entitled “X-ray and Electron Radiotherapy Clinical Treatment Machine;” and

U.S. Pat. No. 4,686,695, to Macovski, entitled “Scanned x-ray Selective Imaging System.”

It should be understood that while the method described was presented with a certain ordering of the steps, it is not our intent to in any way limit the present invention to a specific step order. It should be appreciated that the various steps can be performed in different orders. Further, we have described herein the novel features of the present invention, and it should be understood that we have not included details well known by those of skill in the art, such as the design and operation of a radiation system.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of the appended claims. For example, regardless of the content of any portion (e.g., title, section, abstract, drawing figure, etc.) of this application, unless clearly specified to the contrary, there is no requirement for any particular described or illustrated activity or element, any particular sequence of such activities, any particular size, speed, dimension or frequency, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Accordingly, the descriptions and drawings are to be regarded as illustrative in nature, and not as restrictive.

We claim:

1. A radiation system for irradiating a subject, said system comprising:
a directed charged particle beam source for supplying charged particles;
a scanning device for scanning said charged particles received from said source;
a target, wherein said scanned charged particles impinges upon said target for supplying photons; and
a collimator device, said collimator device collimates said photons, wherein said collimator device is adapted to provide emerging radiation beams to the subject, said emerging radiation beams being sharply forward directed to the subject and with a small cross-section to form beamlets.

2. The system of claim 1, wherein said emerging radiation beams comprises at least one of: high energy x-rays, low energy x-rays, protons, and electrons.

3. The system of claim 1, wherein said collimator attenuates at least some non-forward components of said emerging radiation beams.

4. The system of claim 1, wherein said collimator attenuates all non-forward components of said emerging radiation beams.

5. The system of claim 1, wherein said subject is a human or an animal.

6. The system of claim 1, wherein said beamlets comprises pencil beams.

7. The system of claim 1, wherein said small cross-section is less than about 50 cm.

8. The system of claim 1, wherein said small cross-section is less than about 5 cm.

9. The system of claim 1, wherein said small cross-section is less than about 5 mm.

10. The system of claim 1, wherein said small cross-section is less than about 0.5 mm.

11. The system of claim 1, wherein said small cross-section is less than about 0.05 mm.

12. The system of claim 1, wherein said small cross-section is less than about 0.005 mm.

13. The system of claim 1, wherein said small cross-section is in less than about 0.001 mm.

14. The system of claim 1, wherein said directed charged particle beam source comprises at least one of:
an accelerator wave guide device and accelerator tube device,
x-ray tube device,
linear accelerator device,
betatron device,
race track microtron device, and
laser device.

15. The system of claim 1, wherein said scanning device comprises an electromagnetic apparatus.

16. The system of claim 15, wherein said electromagnetic apparatus comprises a one-dimensional electromagnetic apparatus.

17. The system of claim 15, wherein said electromagnetic apparatus comprises a two-dimensional electromagnetic apparatus.

18. The system of claim 15, wherein said electromagnetic apparatus comprises a three-dimensional electromagnetic apparatus.

19. The system of claim 1, wherein said collimator device comprises a dual focused collimation grid.

20. The system of claim 19, wherein said collimator device comprises a passive dual focused collimation grid.

21. The system of claim 1, further comprising:
a treatment planning system (TPS) that yields a series of optimized intensity maps corresponding to the relative anatomical geometry that exists at a given point in a respiratory or cardiac cycle.

22. The system of claim 21, wherein said treatment planning system (TPS) comprises deformable anatomical models and a four dimensional imaging modality

23. The system of claim 1, further comprising a control unit for generating signals for controlling said radiation system.

24. The system of claim 23, wherein said signals from said control unit causing said beamlets to be provided in an appropriate intensity map in relation to respiratory feedback regarding the subject.

25. The system of claim 24, wherein said signals from said control unit causing said beamlets to be provided in an appropriate intensity map in relation to feedback regarding the subject.

26. The system of claim 25, wherein said feedback regarding the subject includes organ data.

27. The system of claim 26, wherein said organ data includes cardiac data.

28. The system of claim 23, wherein the subject includes a plurality of structures, and wherein said signals from said control unit causing said beamlets to irradiate desired structures of said plurality of structures of the subject.

29. The system of claim 23, wherein the subject includes a plurality of structures, and wherein said signals from said control unit causing said beamlets to avoid irradiating select structures of said plurality of structures of the subject.

30. The system of claim 23, wherein said signals from said control unit causing said beamlets to be modulated in doses wherein increment of said doses are less than duration of a respiratory cycle of the subject.

31. The system of claim 30, wherein said signal from said control unit causing said beamlets to be modulated according to the motion of the patient motion in real time and deliver an optimized radiation pattern.

32. The system of claim 31, wherein said signal from said control unit causing said beamlets to be modulated according to the motion of the patient motion in delay time and deliver an optimized radiation pattern.

33. The system of claim 23, wherein said signals from said control unit causing said beamlets to provide modulated radiation fields upon the subject.

34. The system of claim 33, wherein said radiation fields are summed to produce a full dose of fully modulated intensity maps.

35. The system of claim 34, wherein said intensity maps are produced in less than about 5,000 ms.

36. The system of claim 34, wherein said intensity maps are produced in less than about 500 ms.

37. The system of claim 34, wherein said intensity maps are produced in less than about 50 ms.

38. The system of claim 34, wherein said intensity maps are produced in less than about 5 ms.

39. The system of claim 34, wherein said intensity maps are produced in less than about 100 minutes.

40. The system of claim 34, wherein said intensity maps are produced in less than about 10 minutes.

41. The system of claim 34, wherein said intensity maps are produced in less than about 1 minute.

42. The system of claim 33, wherein said signals from said control unit causing said beamlets to be provided in an appropriate intensity map in relation to respiratory feedback regarding the subject.

43. The system of claim 33, wherein the subject includes a plurality of structures, and wherein said signals from said control unit causing said beamlets to irradiate desired structures of said plurality of structures of the subject.

44. The system of claim 33, wherein the subject includes a plurality of structures, and wherein said signals from said control unit causing said beamlets to avoid irradiating select structures of said plurality of structures of the subject.

45. The system of claim 23, wherein said signals from said control unit causing said beamlets to be provided serially in an appropriate intensity map in relation to respiratory feedback regarding the subject.

46. The system of claim 45, wherein the respiratory feedback is provided from a spirometry device or fluoroscopy device.

47. The system of claim 45, wherein the respiratory feedback is provided from at least one of:
a spirometry device,
optical tracking device adapted to track infrared emitters on the subject,
fluoroscopy device adapted to track implanted markers on the subject, and
direct tracking of targets in the subject, wherein the direct tracking is provided by real time imaging techniques.

48. The system of claim 45, wherein the serially provided beamlets are provided during select periods of the respiratory cycle of the subject.

49. The system of claim 45, wherein the subject includes a plurality of structures, and wherein said signals from said control unit causing said beamlets to irradiate desired structures of said plurality of structures of the subject.

50. The system of claim 45, wherein the subject includes a plurality of structures, and wherein said signals from said control unit causing said beamlets to avoid irradiating select structures of said plurality of structures of the subject.

51. The system of claim 50, wherein said select structures include critical structures.

52. The system of claim 51, wherein said critical structures include at least one of spinal chord, bowel, lung, heart, bronchi, esophagus and trachea.

53. A radiation system for irradiating a subject, said system comprising:
a directed charged particle beam source for supplying charged particles;
a scanning device for scanning said charged particles received from said source; and
a collimator device, said collimator device receives said charged particles received from said scanning device and collimates said charged particles as electrons, wherein said collimator device is adapted to provide emerging radiation beams to the subject, said emerging radiation beams being sharply forward directed to the subject and with a small cross-section to form beamlets.

54. A method for irradiating a subject, said method comprising:
supplying charged particle beams;
scanning said charged particles received from said source;
converting said scanned charged particles into photons; and
collimating said photons to provide emerging radiation beams to the subject, said emerging radiation beams being sharply forward directed to the subject and with a small cross-section to form beamlets.

55. The method of claim 54, further comprising:
providing a treatment planning that yields a series of optimized intensity maps corresponding to the relative anatomical geometry that exists at a given point in a respiratory or cardiac cycle.

56. The method of claim 55, wherein said treatment planning comprises deformable anatomical models and a four dimensional imaging modality

57. The method of claim 54, comprising generating signals for controlling said radiation method.

58. The method of claim 57, wherein said control signals causing said beamlets to be provided in an appropriate intensity map in relation to respiratory feedback regarding the subject.

59. The method of claim 58, wherein said control signals causing said beamlets to be provided in an appropriate intensity map in relation to feedback regarding the subject.

60. The method of claim 59, wherein said feedback regarding the subject includes organ data.

61. The method of claim 60, wherein said organ data includes cardiac data.

62. The method of claim 57, wherein the subject includes a plurality of structures, and wherein said control signals causing said beamlets to irradiate desired structures of said plurality of structures of the subject.

63. The method of claim 57, wherein the subject includes a plurality of structures, and wherein said control signals causing said beamlets to avoid irradiating select structures of said plurality of structures of the subject.

64. The method of claim 57, wherein said control signals causing said beamlets to be modulated in doses wherein increment of said doses are less than duration of a respiratory cycle of the subject.

65. The method of claim 64, wherein said control signals causing said beamlets to be modulated according to the motion of the patient motion in real time and deliver an optimized radiation pattern.

66. The method of claim 65, wherein said control signals causing said beamlets to be modulated according to the motion of the patient motion in delay time and deliver an optimized radiation pattern.

67. The method of claim 57, wherein said control signals causing said beamlets to provide modulated radiation fields upon the subject.

68. The method of claim 67, wherein said radiation fields are summed to produce a full dose of fully modulated intensity maps.

69. The method of claim 67, wherein said control signals causing said beamlets to be provided in an appropriate intensity map in relation to respiratory feedback regarding the subject.

70. The method of claim 67, wherein the subject includes a plurality of structures, and wherein said signals from said control unit causing said beamlets to irradiate desired structures of said plurality of structures of the subject.

71. The method of claim 67, wherein the subject includes a plurality of structures, and wherein said signals from said control unit causing said beamlets to avoid irradiating select structures of said plurality of structures of the subject.

72. The method of claim 54, wherein said control signals causing said beamlets to be provided serially in an appropriate intensity map in relation to respiratory feedback regarding the subject.

73. The method of claim 72, wherein the serially provided beamlets are provided during select periods of the respiratory cycle of the subject.

74. The method of claim 72, wherein the subject includes a plurality of structures, and wherein said signals from said control unit causing said beamlets to irradiate desired structures of said plurality of structures of the subject.

75. The method of claim 72, wherein the subject includes a plurality of structures, and wherein said signals from said control unit causing said beamlets to avoid irradiating select structures of said plurality of structures of the subject.

76. The method of claim 75, wherein said select structures include critical structures.

77. The method of claim 76, wherein said critical structures include at least one of spinal chord, bowel, lung, heart, bronchi, esophagus and trachea.

78. A method for irradiating a subject, said method comprising:
supplying charged particle beams;
scanning said charged particles received from said source; and
collimating said charged particles as electrons to provide emerging radiation beams to the subject, said emerging radiation beams being sharply forward directed to the subject and with a small cross-section to form beamlets.
